(19) United States
(12) Patent Application Publication
(10) Pub. No.: US 2012/0156771 A1
(43) Pub. Date: Jun. 21, 2012

(54) FUNCTIONAL DISRUPTION OF AN IMMUNOGLOBULIN GENE IN AN AVIAN EMBRYONIC STEM CELL

(75) Inventors: Robert J. Etches, San Mateo, CA (US); Robert M. Kay, San Francisco, CA (US); Lei Zhu, Palo Alto, CA (US)

(73) Assignee: Origen Therapeutics, Inc.

(21) Appl. No.: 13/281,362

(22) Filed: Oct. 25, 2011

Related U.S. Application Data
(62) Division of application No. 12/896,681, filed on Oct. 1, 2010, now abandoned.
Application No. 12/896,681 is a continuation of application No. 11/977,538, filed on Oct. 24, 2007, now abandoned.
Application No. 11/977,538 is a continuation of application No. 10/104,486, filed on Mar. 22, 2002, now abandoned.

(51) Int. Cl. C12N 5/10

(52) U.S. Cl. 435/349

(57) ABSTRACT

A transgenic chicken is disclosed having disrupted endogenous immunoglobulin production. In one embodiment, a targeting construct is stably integrated into the genome of the chicken by homologous recombination in embryonic stem cells, and injection of the engineered embryonic stem cells into recipient embryos, thereby knocking out the endogenous immunoglobulin gene locus in resulting animals. The targeted disruption of the locus in embryonic stem cells is particularly useful in combination with the insertion of genetic elements encoding exogenous immunoglobulin molecules. After these chickens are cross-bred, a line of chickens is produced that has a reduction of endogenous immunoglobulin molecule production.

### RELATED INFORMATION

This application is a divisional of application Ser. No. 12/896,681 filed Oct. 1, 2010, which is a continuation of application Ser. No. 11/977,538 filed on Oct. 24, 2007, which is a continuation of application Ser. No. 10/104,486 filed on Mar. 22, 2002, now abandoned. The priority of the prior application is expressly claimed, and the disclosure of each of these prior applications is hereby incorporated by reference in its entirety.

### FIELD OF THE INVENTION

This invention relates to the fields of genetic engineering and non-mammalian transgenic animals. In particular, this invention relates to avians having a functional disruption of endogenous immunoglobulin production, constructs to disrupt the immunoglobulin gene, related cell lines and compositions, and methods for disrupting avian immunoglobulin genes.

### BACKGROUND OF THE INVENTION

The two major components of the vertebrate immune system are B and T lymphocytes. The B cells are responsible for producing very specific proteins called “antibodies,” or “immunoglobulins,” that form part of the immune response that protects an organism from immunogenic substances referred to as antigens. Immunoglobulins are large molecules composed of two identical light (L) polypeptide chains and two identical heavy (H) chains, held together by disulphide bonds. Each polypeptide chain has a variable (V) and a constant (C) region of amino acid sequences. The variable regions contain portions that are designed by the B-cell to uniquely recognize virtually any antigen and thereby specifically bind to the antigen as part of an immune response.

To produce an effective immune response, the immune system must be able to produce a large number of distinct immunoglobulin molecules to any antigen that may be encountered. However, in their fully mature state, most B cells produce only a single antibody specificity. Thus, an effective immune response requires a population of B cells that is undifferentiated and has the ability to differentiate into a repertoire of B cells with the ability to express specific antibodies to meet the challenge of any antigen. Most vertebrates use a characteristic method of immunoglobulin gene rearrangement to create a diverse repertoire of B lymphocytes capable of producing a diverse repertoire of antibodies. The immunoglobulin gene locus contains multiple functional regions of the gene, including discrete segment called the variable (V), diversity (D) and joining (J) gene segments. These segments are recombined during B cell development, and in response to antigen, to generate a functionally rearranged immunoglobulin gene that express an immunoglobulin molecule chain that, when assembled into an intact antibody molecule, specifically binds an antigen.

Because of their unique ability to bind antigens with a high degree of selectivity and specificity, antibodies are highly useful in both diagnostic and therapeutic applications. However, in therapeutic applications, because the human immune system is capable of identifying antibodies that are produced in a non-human species, and developing an immune response thereto, the development of antibodies for human therapeutic use has faced significant hurdles. One approach to produce antibodies that are more useful for human therapy is to create a transgenic animal containing the functional genetic components of the human immune system. When such animals are challenged with antigen, the animals produce a repertoire of antibodies that are substantially human. To create such animals, selected portions of human immunoglobulin genes have been inserted into the genome of the animal with sophisticated genetic engineering techniques. In addition, separate techniques have been used to disrupt the production of the animal's endogenous immunoglobulins. To eliminate the production of endogenous immunoglobulins, the immunoglobulin gene is functionally disrupted in such a manner that the gene cannot undergo rearrangement to yield a configuration capable of encoding an antibody. Disruption of functional immunoglobulin gene rearrangement accompanies the failure of the B cell population to evolve and differentiate into a repertoire capable of expressing antibodies, particularly high affinity isotypes such as IgG.

There are several techniques to functionally disrupt, or to create gene “knockouts” in a transgenic animal. These methods include homologous recombination between an endogenous gene and a targeting construct, microcell-mediated chromosome transfer to insert a defective gene locus into a genome, and telomere associated chromosome truncation in which a region at the end of a chromosome is removed by insertion of a telomere.

By using homologous recombination technology, exogenous gene sequences are inserted into the genomic DNA of embryonic stem (ES) cells to inactivate or “knockout” the endogenous genes. The technology has been successfully applied to genes in several animals and specifically to immunoglobulin genes in mice. The principles of homologous recombination in ES cells were developed in the 1970s in yeast, where, contrary to the situation in mammalian cells, the majority of recombinations between introduced vector DNA and genomic DNA occur by homologous recombination as opposed to random integration. Homologous recombination in mammalian species between an artificial targeting vector and an endogenous gene was first achieved for the β-globin locus, although at a very low frequency. In 1981, two groups derived pluripotent embryonic stem cell lines from mouse blastocysts and were able to show that ES cells can colonize the germ line of chimeric mice when injected into blastocysts even after a period of cell growth in tissue culture. The alteration of the mouse genome by homologous recombination in ES cells was achieved for the selectable hypoxanthine phosphoribosyl transferase (Hprt) gene locus (Thomas and Capecchi (1987) Cell 51:503-12).

Targeting of non-selectable genes became possible after enrichment strategies for homologous recombination were developed (e.g., the use of selective markers in a positive or positive/negative selection process). Murine immunoglobulin gene loci soon became targets for selective disruption. For example, Krimpenfort et al. U.S. Pat. No. 5,591,669 and Lonberg, Kay U.S. Pat. No. 5,874,299 described genetically engineered mice that were not able to assemble immunoglobulin heavy chain genes as a result of targeted disruption of the endogenous immunoglobulin gene in murine ES cells. Mice with disrupted endogenous immunoglobulin gene loci were used for breeding with transgenic mice that produce human monoclonal antibodies to yield transgenic mice whose immunoglobulin production was exclusively human.

Although the procedure for a targeted gene knockout using homologous recombination in murine ES cells has been well characterized, the effective disruption of immunoglobulin gene loci in non-mammalian animals such as ayes or birds has not been described. The construction of a successful method related to avian species has proven to be challenging because avian species have an embryology and B cell diversity strategy that is different from mammals. First, unlike most mammals, ayes have only limited combinatorial diversity. For example, chickens have only single functional V and J gene segments at both the H and L chain locus. (Funk and Thompsom (1996) Imm. and Dev. Bio. of the Chicken 17-28). In order to generate the varied repertoire necessary for an effective immune response, chicken B cells diversify their immunoglobulin genes during development in the bursa of Fabricius, an organ only found in bird species. The diversification strategy involves a process of somatic gene conversion, a DNA recombination process which involves unidirectional transfer of nucleotide sequence blocks. This gene conversion process for B cell diversity is only found in a few mammalian species. Therefore, the B-cell development pathway, the immunoglobulin gene rearrangement, and the process of cell maturation and evolved antibody specificities are different for birds than for mammals.

### SUMMARY OF THE INVENTION

The present invention includes genetic constructs for disrupting endogenous immunoglobulin production in ayes, methods for making and using the constructs to produce transgenic aves, and transgenic aves lacking endogenous immunoglobulin production. The methods comprise inserting a construct of the invention into a pluripotent cell and transferring the cell into an embryo to yield a chimera. Through breeding, the construct becomes integrated into the germline of a resulting animal and ultimately results in the disruption of the production of endogenous immunoglobulin molecules. The disruption of endogenous immunoglobulin production may occur by targeted disruption of a specific immunoglobulin gene locus, the substantial removal of an immunoglobulin gene locus, or the insertion of an engineered construct that, through ordinary processes of cell division, replaces an intact endogenous locus in an embryonic stem cell or in the resulting animal. The disruption may include the actual deletion of endogenous gene segments or loci, or the insertion of elements, such as a stop codon, to prevent expression of the gene.

In one embodiment of the invention, the non-mammalian species is a bird having disrupted immunoglobulin production such that, when challenged with antigen, essentially no endogenous antibody production results. In another embodiment, the bird may express non-avian immunoglobulin molecules caused by specifically engineered non-avian constructs incorporated into the bird's germline DNA. These constructs may or may not directly affect the disruption of endogenous immunoglobulin production.

In one embodiment, the present invention is a transgenic chicken produced by introducing a targeting construct comprising at least one selectable marker and at least one homologous portion of the chicken immunoglobulin gene into a DT40 cell, disrupting the endogenous immunoglobulin gene in the DT40 cell by homologous recombination, making microcells incorporating a chromosome bearing the disruption from the disrupted DT40 cell, fusing the microcells with chicken embryonic stem (cES) cells, selecting cES cells carrying the targeted immunoglobulin locus and creating a chimeric chicken that contains the disrupted immunoglobulin locus. The disrupted immunoglobulin locus is inherited by donor-derived offspring of the chimeras and is bred to homozygosity using techniques known in the art. Birds that are homozygous for the disrupted immunoglobulin locus produce negligible amounts of the endogenous immunoglobulin.

Also included in the invention are constructs to disrupt the production of endogenous immunoglobulin production in the chicken and, in specific embodiments, the disruption of an endogenous locus or the insertion of a construct comprising a defective locus that is incapable of functional rearrangement of the immunoglobulin genes. Such targeting constructs and methods of their production utilize a transgene comprising a gene targeting vector, preferably a positive-negative selection vector, that targets the endogenous locus by homologous recombination yielding the functional disruption of a selected gene or a class of gene segments encoding a heavy and/or light endogenous immunoglobulin chain gene. Such endogenous gene segments include variable, diversity, joining and constant region gene segments in the heavy chain locus, and variable, joining, or constant region segments in the light chain locus, as well as combinations of these.

As described in further detail below, a preferred embodiment of the invention utilizes a targeting vector comprised of at least one region of homology to the endogenous chicken immunoglobulin locus and one or more markers that identify embryonic stem cells that have been successfully targeted by the vector. After recombination, the endogenous locus may be rendered non-functional by the deletion of elements required for recombination, such as a V, D, J, or C region, or may have the insertion of one or more sequences such as a stop codon that prevents expression of a partially or totally rearranged locus. In this aspect of the invention, the invention comprises the targeted locus itself, with the discrete regions of the locus oriented in a manner defined by the insertion. In a preferred embodiment, a positive-negative selection vector is introduced to an embryonic stem cell of a chicken after which cells are selected where in the positive-negative selection vector has integrated into the genome of the chicken by homologous recombination at a targeting site. After transplantation into embryos and breeding to homozygosity by techniques known in the art, the resultant transgenic chicken is substantially incapable of mounting an immunoglobulin mediated immune response.

In another embodiment, the immunoglobulin heavy chain gene is located at a site that is proximate to the telomere of an identified chromosome. The location of the heavy chain locus at the telomeric end of a chromosome provides the ability to target the locus through homologous or site specific recombination. The proximity to the telomere of the chromosome, and the ability to target this location for the immunoglobulin heavy chain knockout, is a function of the necessity of the region of DNA that is telomeric to the immunoglobulin heavy chain locus. Depending on the organism, if the telomeric DNA is not necessary for the survival of the organism, such that the deletion of all DNA telomeric of the immunoglobulin heavy chain locus results in a non-lethal mutation, then the disruption of the immunoglobulin heavy chain may be achieved by a recombination event that is centromeric to the immunoglobulin heavy chain locus.

In this embodiment, the construct of the invention includes a construct with a recombination site centromeric to a region of DNA comprised of the immunoglobulin heavy chain gene. Thus, the construct may combine with the endogenous locus at a point centromeric to the entire immunoglobulin locus or at a point within the locus that deletes segments necessary for rearrangement such as V, D, or J segments.

In a preferred version of this embodiment, the chromosome is avian chromosome 15 and site specific recombination is achieved at a engineered recombination site centromeric to a portion of the chicken immunoglobulin heavy chain locus and the construct contains a complimentary recombination site attached to a segment of DNA comprised of at least one human immunoglobulin locus. Specifically, the construct is comprised of the human immunoglobulin light chain lamda locus and/or the human immunoglobulin heavy chain locus together with a complementary recombination site for site specific recombination with chicken chromosome 15. In this specific embodiment, a recombination site is first inserted into chicken chromosome 15 centromeric to a portion of the chicken immunoglobulin heavy chain locus.

When the construct containing the recombination site and the human immunoglobulin locus are submitted to conditions causing recombination between the two recombination sites, the construct replaces all of the endogenous DNA that is telomeric to the recombination site. In a preferred embodiment, the recombination sites are Lox sites as described in U.S. Pat. No. 4,959,317, which is specifically incorporated herein by reference, and the recombination conditions are the expression of the Cre recombination enzyme. In this fashion, the chicken immunoglobulin heavy chain gene can be deleted and replaced with a construct of choice. The construct, when integrated into the avian chromosome, may also contain a second recombination site that is telomeric to the unrearranged human immunoglobulin locus. In a preferred configuration, the modified avian chromosome 15 contains a first recombination site centromeric to the human immunoglobulin light lamda locus and a second dissimilar recombination site telomeric of the lamda locus. This configuration is suited for reaction with a second construct containing a portion of DNA comprising an additional unrearranged human immunoglobulin locus, such as the portion of human chromosome 14 comprising the human immunoglobulin heavy chain locus. Placed under conditions suitable for recombination of the second recombination site, the human immunoglobulin heavy chain locus is integrated into avian chromosome 15 at a site telomeric of the human immunoglobulin light chain lamda locus. In a similar fashion, the human immunoglobulin light chain kappa locus may be integrated into avian chromosome 15 in an orientation compatible with the lamda locus.

As described below, the endogenous heavy chain knockout is preferably achieved in a recombination-proficient cell line such as an avian pre-B cell DT40 cell line or equivalent. Where the functional disruption is achieved by direct targeting of the endogenous locus with a homologous recombination type targeting vector, the method may be performed in recombination-proficient cells lines or directly in pluripotent cells exhibiting an ES cell phenotype.

Also included in the invention are the transgenic chickens that result from the methods of this invention, both homozygous and heterozygous, and related animal models.

### DESCRIPTION OF THE FIGURES

FIG. 1 is a diagram of the pCX/GFP/Puro plasmid construct used for transfection of ES cells and the identification of the contribution of ES cell progeny to chimeras.

FIG. 2 is a FACS analysis of non-transfected chicken ES cells (upper panel) and chicken ES cells that have been transfected with the pCX/GFP/Puro construct and grown in the presence of puromycin (lower panel 2). The analysis shows that substantially all of the transfected cells are expressing the transgene.

FIG. 3 is a Southern analysis of ES cells that have been transfected with the pCX/GFP/Puro construct. The difference in the location of the probe in preparations of DNA digested with BamH1, EcoR1 and a combination of the two endonucleases indicates that the transgene is incorporated into the genome at different sites in the cell lines TB01 and TB09.

FIG. 4 is a schematic of pKO scrambler targeting vector used for the functional disruption or knockout of the endogenous avian immunoglobulin genes.

FIG. 5 is a schematic of a construct for the functional disruption or knockout of the endogenous avian immunoglobulin heavy chain gene.

FIG. 6 is a schematic of a construct for the functional disruption or knockout of the endogenous avian immunoglobulin light chain gene.

FIG. 7 is a schematic of an alternate construct for the functional disruption or knockout of the endogenous avian immunoglobulin light chain gene.

FIG. 8 is a schematic of an embodiment of a method of the invention for producing a modified avian chromosome encoding human immunoglobulins from an unrearranged immunoglobulin locus, wherein the chromosome is assembled in a DT40 cell and introduced into a chicken embryonic stem cell.

### DETAILED DESCRIPTION OF THE INVENTION

The bursa of fabricius is an organ that plays an important role in B-cell development in chickens. The bursa is a unique organ, found only in birds, which arises at day 5 of embryonic life. (Weill and Reynaud (1987) Science 238:1094-98). Removal of the bursa during early embryonic development (up to day 17 of incubation) prevents the animal from mounting an immune response to any immunizing antigen. Bursal development involves two phases. The first is the intraembryonic phase which includes the colonization and the growth of about 10⁴ bursal follicles by expansion of their B cell clones. The second phase is the posthatching period which includes the seeding of bursal cells to the periphery and the continuous expansion of the bursal follicles. By four weeks of age a sufficient number of stem cells has migrated out of the bursa as postbursal stem cells, thus installing the mature chicken B cell immune system in the periphery.

In a transgenic animal of the invention exhibiting an absence of functional endogenous immunoglobulin gene rearrangement, the bursa will not develop normally, but will have characteristic abnormalities indicative of the gene disruption. Thus, a homozygous JH region knockout would have a bursa that was not populated with B cells and has no clear follicular structure and is physically much smaller than a normal bursa. The engineered gene disruption dissociates any possible transcriptional/translational start from the constant region coding sequence and results in a complete lack of immunoglobulin production in the homozygous knockout animal.

The chicken IgL locus encodes a single functional V_{L} gene segment separated by 1.8 kb from a single functional J_{L} gene segment. A single C_{L} region is located 2 kb 3′ from the J_{L} segment. The functional V_{L} segment, designated V_{L1}, is split in the leader region by a 125-bp intron, and the promoter region of V_{L1} includes a conserved octomer box 32 bp upstream from the TATA box. In a 22-kb region upstream of V_{L1}, there is a 25 V_{L}-homologous gene segments situated in both transcriptional orientations. All 25 of these V_{L} gene segments are truncated at the 5′ end and lack a leader exon and promoter region. In addition, most, but not all lack a functional recombination signal sequence (heptamer-spacer-nonamer) at the 3′ end and are not capable of V-J rearrangement. These 25 gene segments are designated at V_{L} pseudogenes, ψV_{L} 1-25.

The chicken IgH locus is also restricted in its capacity for combinatorial diversity (see Cell 59, 171-183 (1989) and Eur. J. Immunol. 21, 2661-2670 (1991). The chicken IgH locus consists of a single functional V_{H1} segment located 15 kb 5′ from a single functional J_{H} gene segment, with approximately sixteen D_{H} segments between V_{H1} and J_{H}. There is little sequence variation between germline D_{H} segments, thereby limiting combinatorial diversity. A cluster of 80-100 V_{H} pseudogene segments (ψV_{H}), spanning a region of 60-80 kb, is present 5′ of the functional V_{H1} gene. As in the case of the ψV_{L} segments, the ψV_{H} segments lack a promoter region, leader exon, and recombination signal sequences. Many of the ψV_{H} segments are situated with alternating transcriptional orientation.

The single V and J segments are rearranged by V-D-J recombination during a brief period of early chicken B cell development creating only limited diversity at the junction of the V and J gene segment. Further diversity of the rearranged V gene is acquired during B cell proliferation in the bursa. There, blocks of pseudogene sequences appear in the rearranged V gene, whereas the sequences of the pseudogenes and the unrearranged V gene segment do not change. This non-reciprocal transfer of sequence information from the pseudogenes into the rearranged V gene was named gene conversion in analogy to similar processes in yeast.

The conversion tracts comprise from 10 to more than 120 bp, and a single V gene can receive segments exchanged from up to six different pseudogenes. The number of events increases with the time that the B cells spend in the bursal environment, consistent with the idea that gene conversion occurs in a stochastic manner, with more events accumulating as the number of cell divisions increases. It was estimated that one successful conversion event occurs every 10 to 15 cell divisions.

The frequency of usage of the ψV segments for conversion events appears to depend on a number of variables. First, pseudogene segments proximal to the V gene are used more frequently than distal ones. Second, ψV segments in the antisense orientation are used preferentially over segments in the sense orientation. Finally, sequence homology seems to be important for the reaction, since pseudogenes with greater sequence similarity to the V gene serve more often as donors.

Homologous recombination of the knock-out construct with the endogenous locus yields a gene in which the J region is absent. The absence of the J region prohibits V-D-J recombination and therefore, a rearranged immunoglobulin locus cannot be generated and a functional immunoglobulin cannot be encoded.

When the immunoglobulin gene knockout is achieved with an engineered chromosome, the engineering of the chromosome is preferably performed in a recombination proficient cell prior to insertion in a pluripotent cell, such as an embryonic stem cell, which is then used to create transgenic animals. The limitations on the creation of genetic constructs used in various types of genetic recombinations is an important element in the field of transgenics, and gene targeting and recombination in embryonic stem cells has limitations. For this reason, certain cell types have been isolated that are recombination proficient. One example is the avian pre-B cell line, commonly designated DT40. Recombination proficient cell lines which display an enhanced frequency of homologous recombination with targeting constructs featuring at least two regions of homology flanking a selectable marker. The preferred recombination proficient cell line is the avian DT40 pre-B cell described in U.S. Pat. No. 5,543,319, which is specifically incorporated herein in its entirety. Cells with increased rates of homologous recombination may be identified by known techniques (see Buerstedde and Takeda, Cell 67:179-185 (1991)). DT40 cells are highly efficient in gene targeting recombination events and have been used to modify mammalian genetic loci to study gene expression and regulation. The use of DT40 cells to produce modified human chromosomes is known. Dieken et al., “Efficient modification of human chromosomal alleles using recombination-proficient chicken/human microcell hybrids,” Nature Genetics, Vol. 12 (February 1996).

Stable, long-term cultures of ES cells are necessary to perform the genetic modifications to disrupt endogenous immunoglobulin production in the chicken. The development of chimeric or transgenic avians requires that chicken embryonic stem (cES) cell lines be created that contribute to somatic tissues when injected into a recipient embryo. Specifically, the embryonic stem cell cultures are sustained for an extended length of time during which desirable phenotypes in chimeric animals resulting from the injection of embryonic stem cells can be identified, and during which genetic modifications can be made to the genome of the embryonic stem cell to introduce targeting constructs or other genetic modifications to disrupt endogenous immunoglobulin production. In preferred embodiments, avian embryonic stem cell cultures are maintained for an extended period of time and can be engineered to contain a targeted or diluted immunoglobulin locus such that endogenous immunoglobulin molecule production is reduced or eliminated.

Chicken ES cell lines are derived from stage X embryos that have a large nucleus and contain a prominent nucleolus. These cells are confirmed to be chicken embryonic stem (cES) cells by morphology after long-term culturing and to yield chimeras when injected into recipient embryos. Moreover, the ES cells enable a high degree of contribution to somatic tissues as determined by extensive feather chimerism. Still further, these embryonic stem cells are demonstrated to be transfected with transgenes. The ES cells stably integrate the transgene and selection of transformed cells is enabled. These transformed cells are capable of forming chimeras wherein the transgene is present in the germline and somatic tissue of the chimera. Embryonic stem cell progeny are derivatives of ES cells that differentiate into non-ES cell phenotypes after introduction of the ES cells into recipient embryos and the formation of a chimera. A transgenic chicken is the progeny of a chimera which has been produced from chicken ES cells carrying a transgene which is stably integrated into the genome when cells derived from the transgenic ES cells have incorporated into the germline. The presence of the transgene in somatic tissue is demonstrated in extraembryonic and somatic tissues including the allantois, endoderm, mesoderm, and ectoderm of the transgenic animal and is broadly detected in all tissues and organ types.

Chicken ES cells were derived from one of two crosses: Barred Rock X Barred Rock or Barred Rock X Rhode Island Red. These breeds were selected to obtain a feather marker when testing the developmental potential of cES cells. The cES cells are injected into White Leghorn embryos, which are homozygous dominant at the dominant, white locus. Chimeric chickens resulting from injection of these ES cells display black feathers from the cES cells and white feathers from the recipient embryo.

Initial establishment of the cES cell culture was initiated according to the protocol developed by J. Petitte, see U.S. Pat. No. 5,565,479, which is specifically incorporated herein by reference. At stage X, the embryo is a small round disk, consisting of approximately 40,000-60,000 cells, situated on the surface of the yolk. To retrieve the embryo a paper ring is put on the yolk membrane, exposing the embryo in the middle. The yolk membrane is cut around the ring, which is then lifted off the yolk. The embryo, attached to the ventral side of the ring, is placed under the microscope and the area pellucida isolated from the area opaca using a fine loop.

TABLE 1
cES cell lines derived on either STO feeder cells or
a polyester insert in CES-80 medium. The cultures were
initiated from both single and pooled embryos.

| | | Substrate | |
|---|---|---|---|
| Cell | Donor | used to derive | |
| line | embryo | cES cells | Endpoint of cell line |
| 009 | pooled | STO | Cultured for 3 months, injected & |
| | | | cryopreserved |
| 029 | pooled | insert | Cultured for over 3 months, injected |
| | | | & cryopreserved |
| 31 | pooled | STO | Injected at 4 days |
| 36 | pooled | STO | Injected at 13 days |
| 50 | pooled | STO | Grown for over 8 months, injected & |
| | | | cryopreserved |
| 63b | pooled | insert | Grown for 3 months and cryopreserved |
| 67I | single | insert | Injected at 45 days of culture |
| 307 | pooled | STO | Injected at 15 days and fixed for |
| | | | staining |
| 314 | pooled | STO | Cultured for over 3 months, injected |
| | | | & cryopreserved |
| 317 | pooled | STO | Injected at 12 days and fixed for |
| | | | staining |
| 324A | single | insert | Cultured for over 6 months and |
| | | | injected |
| 328 | single | insert | Cultured for over 6 months, injected |
| | | | & cryopreserved |
| 329 | single | insert | Cultured for 5 months, injected & |
| | | | cryopreserved |
| 330 | single | insert | Cultured for 3 months and |
| | | | cryopreserved |
| 331 | single | 24 w insert | Cultured for over 3 months and |
| | | | terminated |
| 332 | single | 96 w STO | Cultured for 3 months and |
| | | | cryopreserved |
| 333 | single | 12 w insert | Cultured for over 3 months and |
| | | | terminated |
| 334 | single | 12 w insert | Cultured for over 3 months and |
| | | | terminated |
| 335 | single | 96 w insert | Cultured for over 3 months and |
| | | | terminated |

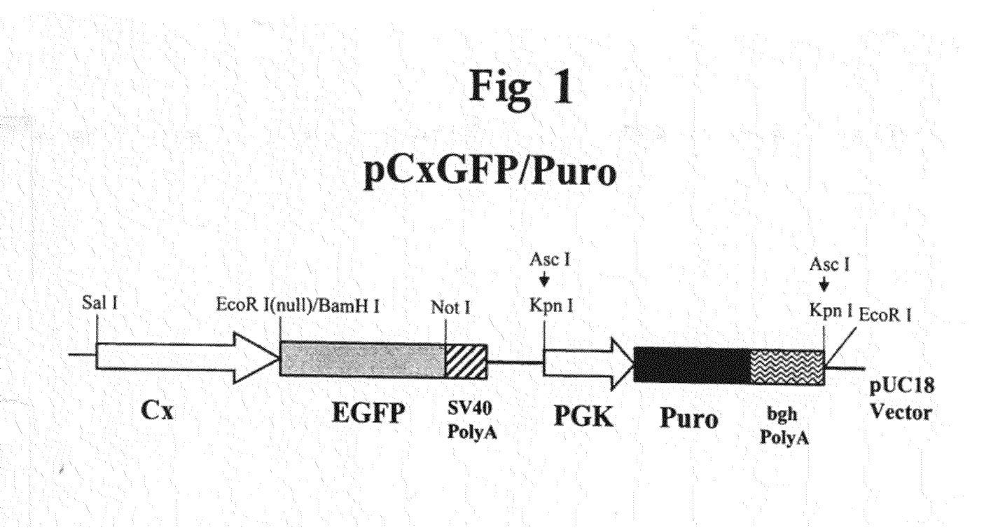

Embryos are dispersed mechanically into a single cell suspension and seeded on a confluent feeder layer of mitotically inactivated STO cells at a concentration of 3×10⁴ cells/cm². The cES culture medium consists of DMEM (20% fresh medium and 80% conditioned medium) supplemented with 10% FCS, 1% pen/strep; 2 mM glutamine, 1 mM pyruvate, 1× nucleosides, 1× non-essential amino acids and 0.1 mM β-mercaptoethanol. Before use, the DMEM medium is conditioned on Buffalo Rat Liver (BRL) cells. Briefly, after BRL cells are grown to confluency, DMEM containing 5% serum is added and conditioned for three days. The medium is removed and a new batch of medium conditioned for three days and repeated. The three batches are combined and used to make the cES medium. Chicken ES cells become visible 3-7 days after seeding of the blastodermal cells. These cES cells were morphologically similar to mES cells; the cells were small with a large nucleus and a pronounced nucleolus (See FIG. 1).

The growth characteristics of cES cells are different from mES cells, which grow in tight round colonies with smooth edges and individual cells that are difficult to distinguish. Chicken ES cells grow in single layer colonies with clearly visible individual cells. Tight colonies are often the first sign of differentiation in a cES culture.

To test for markers of pluripotency of the cells that were derived in culture, the cells were fixed and stained with SSEA-1 (Solter, D. and B. B. Knowles, Proc. Natl. Acad. Sci, U.S.A. 75: 5565-5569, 1978), EMA-1, which recognize epitopes on primordial germ cells in mice and chickens (Hahnel, A. C. and E. M. Eddy, Gamete Research 15: 25-34, 1986) and alkaline phosphatase (AP) which is also expressed by pluripotential cells. The results of these tests demonstrate that chicken ES cells express alkaline phosphatase and the antigens recognized by SSEA-1 and EMA-1.

Although cES cells are visible after using the above protocol, such cultures cannot be maintained longer than a few weeks. Several modifications in culture conditions were initiated, as discussed below, which led to the derivation of 19 cell lines (Table 1) of which 14 were tested for their developmental potential by injection into recipient embryos. Eleven of the 14 cell lines contributed to recipient embryos as determined by feather pigmentation (See Table 2 below). This protocol yields sustained cultures of pluripotent cells expressing an embryonic stem cell phenotype. At any point, the cells can be cryopreserved and when injected into compromised recipient embryos have the potential to substantially contribute to somatic tissues.

TABLE 2
Passage number and time in culture of embryonic stem cell lines derived
from single or pooled embryos. Frequency and extent of somatic chimerism
after injection of these cES cells into stage X recipients.

| | | | time in | # of | | | | Extent of |
|---|---|---|---|---|---|---|---|---|
| Cell | Donor | Passage | culture | embryos | | # | % | chimerism¹ |
| line | embryo | number | (days) | injected | # chimeras | analyzed | chimeras | (%) |
| 31 | pooled | 0 | 4 | 15 | 2 | 7 | 28.5 | 1-5 |
| 317 | pooled | 4 | 12 | 29 | 2 | 10 | 20 | 25-65 |
| 36 | Pooled | 1 | 13 | 24 | 1 | 5 | 20 | 15 |
| 307 | pooled | 4 | 15 | 21 | 1 | 6 | 17 | 5 |
| 330 | single | 6 | 33 | 11 | 3 | 8 | 25 | 5-50 |
| 63b | pooled | 11 | 72 | 36 | 4 | 21 | 19 | 1-10 |
| 67I | single | 3 | 45 | 28 | 0 | 15 | 0 | — |
| 324A | single | 10 | 65 | 25 | 0 | 15 | 0 | — |
| 009 | pooled | 20 | 61 | 27 | 0 | 9 | 0 | — |
| 329 | single | 3 | 15 | 31 | 8 | 17 | 47 | 3-75 |
| 329 | | 6 | 25 | 30 | 9 | 19 | 47 | 3-95 |
| 329 | | 6 | 28 | 26 | 1 | 12 | 8 | 23 |
| 329 | | 11 | 49 | 10 | 1 | 4 | 25 | 60 |
| 029 | pooled | 4 | 33 | 40 | 9 | 27 | 33 | 5-80 |
| 029 | | 9 | 37 | 40 | 4 | 15 | 27 | 4-15 |
| 328 | Single | 6 | 56 | 19 | 4 | 11 | 36 | 10-80 |
| 328 | | 12 | 98 | 33 | 7 | 22 | 32 | 5-50 |
| 314 | Pooled | 17 | 52 | 30 | 2 | 5 | 40 | 5-65 |
| 314 | | 15-17 | 53 | 29 | 1 | 4 | 25 | 30 |
| 314 | | 17 | 55 | 37 | 3 | 15 | 30 | 3-80 |
| 314 | | 16 | 65 | 27 | 2 | 11 | 18 | 5-40 |
| 314 | | 14 | 61 | 25 | 0 | 13 | 0 | — |
| 314 | | 16 | 65 | 32 | 3 | 14 | 21 | 10-60 |
| 314 | | 20 | 61 | 30 | 4 | 5 | 80 | 4-50 |
| 314 | | 21 | 67 | 30 | 2 | 11 | 18 | 5-15 |
| 314 | | 21 | 71 | 8 | 0 | 2 | 0 | — |
| 50 | pooled | 7 | 53 | 35 | 7 | 23 | 30 | 4-65 |
| 50 | | 14 | 106 | 36 | 3 | 21 | 14 | 10-30 |

¹Extent of chimerism was determined by the proportion of black feathers.

As with the mouse, avian embryonic stem cells, which are sometimes referred to as embryonic germ cells, are derived on a variety of feeder layers including STO, STO-sn1 and others that are readily available. Leukemia Inhibitory Factor (LIF) produced by these feeders, and the addition of fetal bovine serum contributes to the maintenance of ES cells in an undifferentiated state. In a preferred embodiment of this invention, chicken ES cell cultures are initiated on a STO feeder layer. STO cells are grown to confluency, treated with 10 μg/ml mitomycin for 3-4 hours, washed, trypsinized and seeded on gelatin coated dishes at 4×10⁴ cells/cm². cES cells appear to grow more rapidly when the feeder of STO cells are sparser. Reducing the STO feeder cell concentration to between 10³ and 10⁵, and preferably below 10⁴ cells/cm², facilitates the derivation and propagation of cES cells. However, when chicken embryonic fibroblasts and mouse primary fibroblasts are used as feeders, no cES cells were derived. Also, when previously established cES cells were plated on these feeders, all of them differentiated within 1 week.

Growing cES cells on synthetic inserts, such as polymer membranes (Costar, Transwell type) in the absence of feeders avoids contamination of the recipient embryo with feeder cells when the ES cells are injected. As Table 3 and 4 show, culturing on inserts, instead of STO feeders, facilitates the derivation of cES cells, and inserts can be used for initial derivation. However, after initially growing rapidly on inserts, the mitotic activity of the ES cells declines after 4-6 weeks of culture. To extend the culture the cells have to be transferred to a feeder of STO cells.

TABLE 3
Establishment of cES cells from single embryos on either
inserts or a feeder of STO cells (10⁴ cells/cm²).

| | # of cultures | # of cell lines |
|---|---|---|
| Substrate | started | obtained |
| STO feeder | 56 | 3 (5%) |
| insert | 45 | 7 (16%) |

TABLE 4
Establishment of cES cells from pooled embryos
on either a STO feeder or a synthetic insert.

| | # of cultures | # of cell lines |
|---|---|---|
| Substrate | started | obtained |
| STO feeder | 73 | 7 (9.5%) |
| insert | 17 | 2 (12%) |

The data in Tables 3 and 4 show that chicken embryonic feeder cells and mouse primary fetal fibroblasts do not support the derivation or the culture of cES cells. A feeder of STO cells supports derivation and growth but only when present in a limited concentration of between 10³ and 10⁵ STO cells but preferably in the present embodiment at a concentration of less than or appropriately 10⁴ cells/cm². A dense STO feeder layer impairs the growth of cES cells, while the specified concentration of STO cells provides factor(s) necessary for ES cell proliferation. When the cells are sustained over an extended culture period and continue to express an embryonic stem cell phenotype, and differentiate into non-embryonic stem cell phenotypes in vivo, they are referred to as “ES cell progeny.”

The cES cell culture medium consists of 80% conditioned medium and preferably contains certain BRL conditioned medium with factors necessary for the derivation and growth of cES cells. At a concentration of 50%, growth of the cES cells is not as reliable as in 80% conditioned medium. When the percentage of conditioned medium is reduced to less than 50%, the growth of the cES cells is affected, as evidenced by an increase in differentiated cells and, at a concentration of 30% or less, the cES cells differentiate within 1 week. This conditioned medium found necessary for the derivation and maintenance of cES cells does not maintain mES but causes their differentiation.

Fetal bovine serum is a preferred component of the ES cell medium according to the present invention and contains factors that keep cES cells in an undifferentiated state. However, serum is also known to contain factors that induce differentiation. Commercially available serum lots are routinely tested by users for their potential to keep ES cells in an undifferentiated state. Serum used for cES cell cultures are known to differ from serum used for mouse ES cell cultures. For example, serum used for the culture of mouse ES cells that is low in cytotoxin and hemoglobin concentration, which is known to maintain mouse ES cells in an undifferentiated state, did not support the sustained growth of chicken ES cells.

Therefore, serum to be used on chicken ES cells should not be tested on mouse ES cells to determine suitability as a media component, but instead should be evaluated on chicken ES cells. To do so, chicken ES cell cultures are divided into two and used to test each new batch of serum. The new batch tested must clearly support the growth of chicken ES cells when empirically tested.

Chicken chromosomal spreads require special evaluation techniques different than mice because the complex karyotype consisting of 10 macrochromosomes, 66 micro-chromosomes and a pair of sex chromosomes (ZZ in males and ZW in females). Long-term cES cells analyzed after 189 days in culture, and after being cyopreserved twice, exhibit a normal karyotype with 10 macro chromosomes; 2 Z-chromosomes and 66 microchromosomes.

Chicken ES cells are cryopreserved in 10% DMSO in medium. After thawing and injecting several cell lines into recipient embryos, somatic chimeras are obtained, indicating that the cES cells maintain their developmental potential during the cryopreservation process.

To permit access to the embryo in a freshly laid egg the shell must be breeched, inevitably leading to a reduction in the hatch rate at the end of the 21-day incubation period. The convention was to cut a small hole (less than 10 mm diameter) in the side of the egg, through which the embryo was manipulated, and re-seal with tape, a glass cover slip, shell membrane or a piece of shell. Though relatively simple to perform, this “windowing” method caused embryonic mortality between 70 and 100%. Improved access to the embryo and increased survival and hatchability can be achieved if the embryo is transferred to surrogate eggshells for incubation to hatching using two different shells and a method (adapted from Callebaut) (Callebaut, Poult. Sci 60: 723-725, 1981) and (Rowlett, K. and K. Simkiss, J. Exp. Biol. 143: 529-536, 1989), which are specifically incorporated herein by reference with this technique, the mean hatch rate is approximately 41% (range 23-70%) with 191 chicks hatched from 469 cES-cell injected embryos.

Incubation of embryos following injection of donor ES cells into recipient embryos can be divided into two parts comprising System A and System B as described below:

System A covers the first three days of post-oviposition development. Fertile eggs containing the recipient embryos are matched with eggs 3 to 5 grams heavier. A 32 mm diameter window is cut at the pointed pole, the contents removed and the recipient embryo on the yolk, together with the surrounding albumen, is carefully transferred into the surrogate shell.

Cells are taken up in a sterile, finely tapered glass pipette connected to a mouth aspirator fitted with a 2 micron filter. The opening of the pipette can be from 50 to 120 microns in diameter and possesses a 30° spiked bevel. The embryo is visualized under low magnification and with blue light. Chicken ES cells are trypsinized into a single cell suspension and 4,000 to 26,000 cells are injected into an embryo. The cells are gently expelled into the space either below or above the embryo, i.e. into the sub-embryonic cavity or between the apical surface of the area pellucida and the perivitelline layer (yolk membrane). Extra albumen collected from fresh fertile eggs is added and the shell sealed with Saran Wrap plastic film.

System B covers the period from day three to hatching. At day three of incubation the embryo has reached around stage 17 (H&H). Water has been transported from the albumen to the sub-embryonic cavity, causing the yolk to enlarge and become very fragile. The contents of the System A shell are very carefully transferred to a second surrogate shell (usually a turkey egg) 30 to 35 grams heavier than the original egg. Penicillin and streptomycin are added to prevent bacterial contamination and the 38 to 42 mm window in the blunt pole is sealed with plastic film. This larger shell allows for an artificial airspace. At day 18 to 19 of incubation the embryo cultures are transferred to tabletop hatchers for close observation. As lung ventilation becomes established, holes are periodically made in the plastic film to allow ambient air into the airspace. Approximately 6-12 hours before hatching the film is replaced with a small petri dish, which the chick can easily push aside during hatching.

For incubation, conventional temperature (37.5 to 38° C.) and relative humidity (50 to 60%) are maintained for the embryos in surrogate shells, but periodic egg rocking, which is normally hourly and through 90 degrees, has to be modified to ensure good survival. In System A rocking is through 90° every 4 to 5 minutes; in System B it is through 40 to 60° every 40 to 45 minutes. In both systems the speed of rocking is maintained at 15 to 20° per minute.

The contribution of cES cells to chimeras is improved if the recipient embryo is prepared by (1) irradiated by exposure to 660 rads of gamma irradiation (2) altered by mechanically removing approximately 1000 cells from the center of the embryo, or by combining (1) and (2) above before the injection of the cES cells. Referring to Table 5, contribution of cES cells to the somatic tissues increased substantially when recipient embryos were compromised, either by removing cells from the center of the recipient embryo or by exposure to irradiation. When the recipient embryos are compromised by a combination of irradiation and mechanical removal of the cells, the contribution of the ES cells is increased further, even though the cES cells had been in culture for longer periods of time. Some of the resulting chimeric chicks are indistinguishable from pure Barred Rock chicks. As the data in Table 5 show, chimerism rates as well as the extent of chimerism per embryo increases after compromising the recipient embryo.

TABLE 5
Frequency of somatic chimerism after injection of cES cells into
recipient embryos that were compromised by different methods.

| | | | | | | Frequency | Extent |
|---|---|---|---|---|---|---|---|
| | # | | | | [symbol] bryos | of | feather |
| Treatment to compromise | Cell | Time cells | | # | & chicks | chimerism | chimerism |
| the recipient embryo | lines | in culture | | Chimeras | evaluated | % | (%) |
| None | 14 | 4-106 | days | 83 | 347 | 24 | 26 |
| Mechanical removal of cells | 1 | 6 | months | 34 | 63 | 54 | 20 |
| Irradiation | 1 | 6-7 | months | 56 | 95 | 59 | 29 |
| Irradiation & Mechanical | 1 | 7-8 | months | 52 | 59 | 88 | 49 |
| removal of cells | | | | | | | |

[symbol]

indicates data missing or illegible when filed

Recipient embryos substantially younger than stage X may also be used to produce chimeras using ES cell as the donor. Early stage recipient embryos are retrieved by injecting the hens with oxytocin to induce premature oviposition and fertile eggs are retrieved at stages VII to IX.

Alternatively, the retrieval of embryos from the magnum region of the oviduct provides access to stage I to VI embryos, consisting of approximately 4-250 cells, and enables the development of chimeras from all embryonic stages as potential recipient embryos.

Transfection of cES cells may be achieved by lipofection and electroporation. Referring to Table 6, an appropriate amount of DNA compatible with the size of the well being transfected is diluted in media absent of serum or antibiotics. The appropriate volume of Superfect (Stratagene) is added and mixed with the DNA, and the reaction is allowed to occur for 5-10 minutes. The media is removed and the wells to be transfected are washed with a Ca/Mg free salt solution. The appropriate volume of media, which can contain serum and antibiotics, is added to the DNA/superfect mixture. The plates are incubated for 2-3 hours at 37 C. When the incubation is completed, the Superfect is removed by washing the cells 1-2× and fresh culture media is added.

TABLE 6
Conditions for transfection of chicken ES cells using Superfect.

| | | Volume of | | | | | Time to | Volume of | | |
|---|---|---|---|---|---|---|---|---|---|---|
| | | media used | | Total | | | form | media | | |
| | | to dilute | | amount of | ul | | complex | added to | | Incubation |
| Plate Size | | DNA | | DNA | Superfect | | (min) | complex | | time |
| 96 | well | 30 | ul | 1 | 5 | ul | 5-10 | 150 | | 2-3 hrs |
| 48 | well | 50 | ul | 1.5 | 9 | ul | 5-10 | 250 | ul | 2-3 hrs |
| 24 | well | 60 | ul | 2 | 10 | ul | 5-10 | 350 | ul | 2-3 hrs |
| 12 | well | 75 | ul | 3 | 15 | ul | 5-10 | 400 | ul | 2-3 hrs |
| 6 | well | 100 | ul | 4 | 20 | ul | 5-10 | 600 | ul | 2-3 hrs |
| 60 | mm | 150 | ul | 10 | 50 | ul | 5-10 | 1000 | ul | 2-3 hrs |
| 100 | mm | 300 | ul | 20 | 120 | ul | 5-10 | 3000 | ul | 2-3 hrs |

A petri-pulser is used to electroporate cES cells that are attached to the plate in a 35 mm diameter well. The media is removed and the well is washed with a salt solution without Ca⁺⁺ and Mg⁺⁺. One ml of electroporation solution is added to the well. DNA is added and the media is gently mixed. The petri-pulser is lowered onto the bottom of the well and an electrical current is delivered. (Voltage preferably varies from 100-500 V/cm and the pulse length can be from 12-16 msec). The petri-pulser is removed and the electroporated well is allowed to stand for 10 minutes at room temperature. After 10 minutes, 2 mls of media is added and the dish is returned to the incubator.

To transfect cells in suspension, media is removed and cells are washed with a Ca/Mg free salt solution. Tryspin with EDTA is added to obtain a single cell suspension. Cells are washed, centrifuged and resuspended in a correctional electroporation buffer solution such as PBS. The ES cell suspension is placed into a sterile cuvette, and DNA added (minimum concentration of 1 mg/ml) to the cell suspension and mixed by pipetting up and down. The cells are electroporated and allowed to sit at RT for 10 minutes. Cells are removed from cuvette and distributed to previously prepared wells/dishes. Cells are placed in an incubator and evaluated or transient transfection 24-48 hours after electroporation. Selection of antibiotic resistant cells may also be started by including an antibiotic such as puromycin in the culture medium.

In a preferred embodiment, the concentration of puromycin required for selecting transfected cells is calculated as a titration kill curve. Titration kill curves for chicken embryonic stem cells are established by exposing cells in culture to puromycin concentrations varying from 0.0 to 1.0 μg/ml for 10 days (Table 7) and neomycin concentrations varying from 0.0 to 200 μg/ml (Table 8). The medium is changed every 2 days and fresh puromycin or neomycin is added. When exposed to a concentration of 0.3 μg/ml puromycin, ES cells were absent from all wells after 3 changes of medium with fresh puromycin over a six day period (see Table 7). Puromycin concentrations of 0.3-1.0 μg/ml are used for selection of the transfected cultures. Neomycin concentrations over 40 μg/ml eliminated all cES cells within 7 days (Table 8).

After 10 days of selection, cES cells colonies are visible and can be picked for further expansion.

TABLE 7
Morphology of cES cells after exposure of various concentrations of puromycin
and different lengths of time (days after addition of puromycin).

| Puromycin conc. | Time under selection (days) | | | | | | | | | |
|---|---|---|---|---|---|---|---|---|---|---|
| (μg/ml) | 1 | 2 | 3 | 4 | 5 | 6 | 7 | 8 | 9 | 10 |
| 0.0 | ES | ES | ES | ES | ES | ES | ES | ES | ES | ES |
| 0.1 | ES | ES | ES | ES | ES | ES | ES | ES | ES | ES |
| 0.2 | ES | ES | ES | ES | ES | ES | ES | ES | ES | ES |
| 0.25 | ES | ES | ES | ES | ES | diff | diff | diff/ | diff/ | diff/ |
| | | | | | | | | gone | gone | gone |
| 0.3 | ES | ES | diff | diff/ | diff/ | gone | gone | gone | gone | gone |
| | | | | gone | gone | | | | | |
| 0.4 | ES | diff | gone | gone | gone | gone | gone | gone | gone | gone |
| 0.5 | diff | gone | gone | gone | gone | gone | gone | gone | gone | gone |
| 0.6 | diff | gone | gone | gone | gone | gone | gone | gone | gone | gone |
| 0.7 | diff | gone | gone | gone | gone | gone | gone | gone | gone | gone |
| 0.8 | gone | gone | gone | gone | gone | gone | gone | gone | gone | gone |

ES: ES cells are present.
diff: ES cells are differentiated.
gone: no morphologically recognizable cells are present

TABLE 8
Morphology of cES cells after exposure of various concentrations of neomycin
and different lengths of time (days after addition of neomycin).

| Neomycin | | | | | | | | | | |
|---|---|---|---|---|---|---|---|---|---|---|
| conc. | Time under selection (days) | | | | | | | | | |
| (μg/ml) | 1 | 2 | 3 | 4 | 5 | 6 | 7 | 8 | 9 | 10 |
| 0.0 | ES | ES | ES | ES | ES | ES | ES | ES | ES | ES |
| 10 | ES | ES | ES | ES | ES | ES | ES | ES | ES | ES |
| 20 | ES | ES | ES | ES | ES | ES | ES | ES | ES | ES |
| 30 | ES | ES | ES | ES | ES | ES | ES/Diff | ES/diff | Diff | Diff/ |
| | | | | | | | | | | gone |
| 40 | ES | ES | ES | ES | ES/Diff | Diff/ | dead | gone | gone | gone |
| | | | | | | dead | | | | |
| 50 | ES | ES | ES | ES/Diff | ES/Diff | Diff/ | Dead/ | gone | gone | gone |
| | | | | | | dead | gone | | | |
| 60 | ES | ES | ES | gone | Gone | gone | gone | gone | gone | gone |
| 100 | ES/Diff | Diff | dead | gone | Gone | gone | gone | gone | gone | gone |
| 150 | | dead | dead | gone | Gone | gone | gone | gone | gone | gone |
| 200 | | dead | gone | gone | Gone | gone | gone | gone | gone | gone |

Selection of transfected chicken ES cells and their identification in chimeras requires that the transgene confer a selective advantage to the cells in culture (e.g. resistance to puromycin in the medium). To analyze the contribution of modified ES cells to the resulting chimera, an identifiable gene product can be included in the transgene incorporated into the ES cells. This can be accomplished using pCX/GFP/Puro which provides resistance to puromycin in cES cells and produces a green fluorescent protein (GFP) in most, if not all, donor-derived cells in chimeras.

Referring to FIG. 1, PCX/GFP/Puro was produced in three cloning steps involving two intermediates before make the final pCX/GFP/Puro plasmid. In step 1, the PGK-driven Puromycin resistant gene cassette (1.5 Kb) was released from pKO SelectPuro (Stratagene) by Asc I digestion. The fragment was then blunted and Kpn I linkers were added. The resulting fragment (GFP/Puro) was inserted into the corresponding Kpn I site of pMIEM (courtesy of Jim Petitte (NCSU), a GFP expression version derived from LacZ expression pMIWZ, see Cell Diff and Dev. 29: 181-186 (1990) to produce the first intermediate (pGFP/Puro). The PGK-Puro cassette was in same transcription orientation as GFP (determined by BamH I and Sty I digestion). In step 2, the GFP/Puro expression cassette (2.5 Kb) was released from pGFP/Puro by BamH I and EcoR I double digestion. The resulting fragment was inserted into the BamH I and EcoR I sites of pUC18 (Invitrogen). It contains 5′ unique sites, Hind III, Pst I and Sal I. The resulting plasmid pUC18/GFP/Puro was verified by a BamH I, EcoR I, and Not I triple digestion. In the third step, the Cx promoter including 384 bp CMV-IE enhancer, 1.3 kb chicken beta-actin promoter and portion of 1^{st} intron was released from pCX-EGFP (Masahito, I. et al., (1995) FEBS Letters 375: 125-128) by Sal I and EcoR I digestion. A 3′ EcoR I (null)-Xmn I-BamH I linker was attached to the fragment and it was inserted into the Sal I and BamH I sites of pUC18/GFP/Puro. The plasmid pCX/GFP/Puro was verified by a BamH I and Pst I double digestion. pCX/GFP/Puro DNA can be linearized by Sca I digestion for transfection into cES cells.

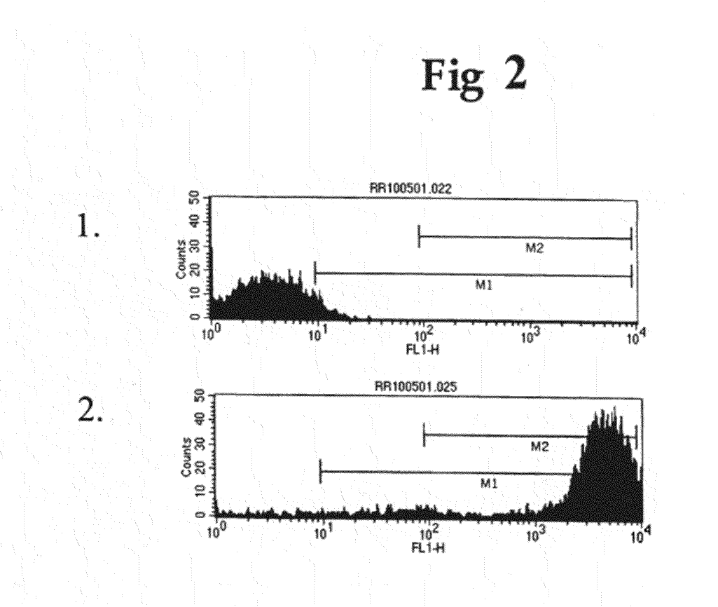

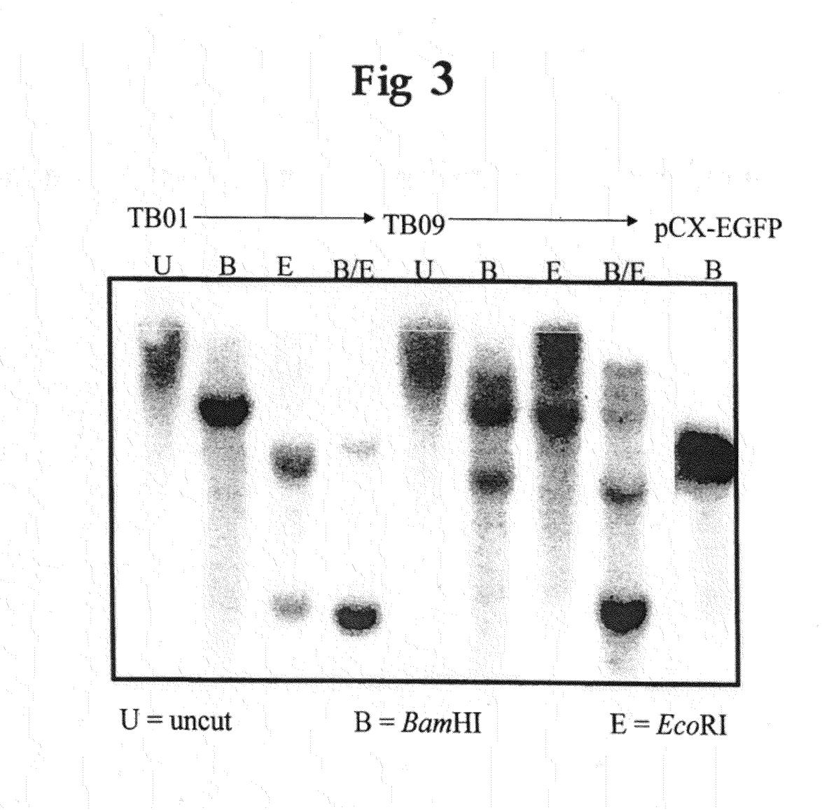

Transfection and selection of ES cells using the procedures described above produced a population of cells that would grow in the presence of 0.5 ug of puromycin. These cells exhibited green fluorescence when examined by conventional fluorescence microscopy. When preparations of the ES cells are examined by fluorescence activated cell sorting, it is evident that essentially all of the cells carry and express the transgene (See FIG. 2). Southern analysis of DNA from the transfected ES cell lines TB01 and TB09 that was digested with BamH1, EcoRI or both restriction endonucleases revealed the transgene in DNA fragments of various sizes, providing evidence that the transgene is integrated into the genome (See FIG. 3).

The CX/GFP/Puro construct demonstrates that transgenes of at least 4.5 kb can be inserted into chimeric chickens. Using the cES cells described herein, chicken ES cells can be transfected with different or larger constructs. As noted above, by using a specially designed targeting constructs, the avian immunoglobulin gene can be functionally disrupted by homologous recombination in ES cells or in a cell line such as the avian pre-B cell line DT40 followed by transfer of the modified locus into a pluripotent cell. Other possible genetic modifications include insertion by the targeting construct of regulatory and/or coding sequences, deletion of one or more nucleotides, and replacement of one or more native nucleotides that may result in a change in amino acid codon(s). Modifications may include replacement of promoter regions, or insertion of upstream inactivation sequences (i.e., stop codons).

The targeting construct may be prepared using common techniques that are well established in the art and include restriction digest and ligation, PCR mutagenesis, and chemical synthesis of suitable oligonucleotides. In an embodiment, the targeting construct is a plasmid. The targeting construct contains regions that are homologous with sequences that are within or which flank the immunoglobulin gene loci. It would be evident to one skilled in the art to apply the knowledge established in the literature to determine the minimum and maximum length of the homologous regions required for homologous recombination in the subject cells. The regions of homology between these recombination site sequences and the target sequences will typically be at least about 90%, usually greater than 95%. The regions of homology are preferably within coding regions, such as exons, of the gene.

As noted above, a selectable marker is incorporated within the targeting construct to facilitate identification of successful integration events. The choice of a suitable selectable marker would be evident to one skilled in the art. Considerations in selected a suitable selectable marker include, but are not limited to, the genotype of the recipient cell (i.e., production of immunoglobulin molecules) and the presence of their selectable markers on the chromosome. Suitable selectable markers include the neomycin resistance gene (neo) and puromycin (puro). Preferably, this invention includes use of a positive selection marker and a negative selection marker.

In one preferred embodiment, the targeting construct places the gene cassette encoding the resistance marker behind the ATG-start codon. For example, introduction of a β-galactosidase (lacZ) gene in-frame with the immunoglobulin gene (or a geo-fusion composed of lacZ and neo^{r}), will not only allow the disruption of the reading frame but will yield additional information about the spatial transcription pattern of the investigated gene.

In another embodiment, the targeting construct comprises a sequence in which the desired sequence modifications are flanked by DNA substantially isogenic with a corresponding target sequences in the chicken immunoglobulin loci to be disrupted. The substantially isogenic sequence is preferably at least about 97-98% identical with the corresponding target sequence (except for the desired sequence modifications), more preferably at least about 99.9-99.5% identical, most preferably about 99.6 to 99.9% identical. The targeting construct and the target DNA preferably share stretches of DNA at least about 75 base pairs that are perfectly identical, more preferably at least about 150 base pairs that are perfectly identical, and even more preferably at least about 500 base pairs that are perfectly identical. Accordingly, it is preferable to use a targeting construct derived from cells as closely related as possible to the cell line being targeted; more preferably, the targeting DNA is derived from cells to the same cell line as the cells being targeted. Most preferably, the target construct is derived from cells of the same chicken as the cells being targeted.

Preferably, the targeting construct sequence is at least about 100-200 bp of substantially isogenic DNA, more preferably at least bout 300-1000 bp and generally less than about 15,000 bp. The amount of targeting DNA present on either side of a sequence modification can be manipulated to favor either single or double crossover events, both of which can be obtained using the present invention. In a double crossover or “replacement-type” event, the portion of the targeting construct between the two crossovers will replace the corresponding portion of the target DNA. In a single crossover or “insertion-type” event, the entire targeting sequence will generally be incorporated into the immunoglobulin gene sequence at the site of the single crossover. To promote double crossovers, the modification sequences are preferably flanked by homologous sequences such that, upon integration, the modification sequences are located towards the middle of the flanking homologous sequences. If single crossovers are desired, the targeting construct should be designed such that the ends of the linearized homologous sequence correspond to target DNA sequences lying adjacent to each other in the genome.

In another embodiment of this invention, a cloned telomeric region is used as a portion of the targeting construct, homologous integration of the construct into the chromosome produces a targeted deletion of an entire chromosome locus and results in the total elimination of the locus that is downstream from the site of the homologous integration. Furthermore, where the cloned telomeric region contains an exogenous gene, such as a human immunoglobulin locus, the construct can be used to create a chimeric chromosome that is comprised of a native chromosome, which may itself have engineered genetic modifications, and an exogenous gene that is telomeric of the site of homologous integration. In a particularly preferred embodiment, the exogenous gene is an unrearranged human immunoglobulin heavy chain locus that is incorporated into the construct between the telomeric region and the region for homologous insertion and includes a selectable marker gene. In this embodiment, the human immunoglobulin heavy chain locus can be targeted into an avian chromosome containing an avian immunoglobulin locus to yield a chimeric chromosome that harbors human immunoglobulin gene DNA but completely lacks the endogenous avian immunoglobulin locus.

Any technique that can be used to introduce DNA into the animal cells of choice can be employed. Electroporation has the advantage of ease and has been found to be broadly applicable, but a substantial fraction of the targeted cells may be killed during electroporation. Therefore, for sensitive cells or cells which are only obtainable in small numbers, microinjection directly into nuclei may be preferable. Also, where a high efficiency of DNA incorporation is especially important, such as targeting without the use of a selectable marker, direct microinjection into nuclei is an advantageous method because typically 5 to 25% of targeted cells will have stably incorporated the microinjected DNA. Retroviral vectors are also highly efficient but in some cases they are subject to other shortcomings. Where lower efficiency techniques are used, such as electroporation, calcium phosphate precipitation or liposome fusion, it is preferable to have a selectable marker in the targeting DNA so that stable transformants can be readily selected. A variety of such transformation techniques are well known in the art, including:
1. Direct Microinjection into the Nuclei

Targeting constructs can be microinjected directly into animal cell nuclei using micropipettes to mechanically transfer the recombinant DNA. This method has the advantage of not exposing the DNA to cellular compartments other than the nucleus and of yielding stable recombinants at high frequency. (See, Capecchi (1980) Cell 22:479-88).
2. Electroporation

The targeting DNA can also be introduced into the animal cells by electroporation. In this technique, animal cells are electroporated in the presence of DNA containing the targeting construct. Electrical impulses of high field strength reversibly permeable biomembranes allowing the introduction of the targeting construct. The pores created during electroporation permit the uptake of macromolecules such as DNA. The procedure is described in, e.g., Potter et al. (1984) Proc. Nat'l. Acad. Sci. U.S.A. 81:7161-65.
3. Calcium Phosphate Precipitation

The targeting constructs may also be transferred into cells by other methods of direct uptake, for example, using calcium phosphate. See, e.g. Graham and Van der Eb (1973) Virology 52:456-67.
4. Liposomes

Encapsulation of DNA within artificial membrane vesicles (liposomes) followed by fusion of the liposomes with the target cell membrane can also be used to introduce DNA into animal cells. See Mannino and Gould-Fogerite (1988) BioTechniques 6:682.
5. Viral Capsids

Viruses and empty viral capsids can also be used to incorporate DNA and transfer the DNA to the chicken cells. For example, DNA can be incorporated into empty polyoma viral capsids and then delivered to polyoma-susceptible cells. See, e.g., Slilaty and Aposhian (1983) Science 220:725.
6. Transfection Using Polybrene or DEAE-Dextran

This transfection technique is described in Sambrook, J. et al. (1989) Chapter 16, In Molecular Cloning. A Laboratory Manual, Cold Spring Harbor Laboratory Press, Cold Spring Harbor, N.Y.
7. Protoplast Fusion

Protoplast fusion typically involves the fusion of bacterial protoplasts carrying high numbers of a plasmid of interest with cultured animal cells, usually mediated by treatment with polyethylene glycol. (Rassoulzadegan et al. (1982) Nature 295:257).
8. Ballistic Penetration

Another method of introduction of nucleic acid segments is high velocity ballistic penetration by small particles with the nucleic acid either within the matrix of small beads or particles, or on the surface. (Klein et al. (1987) Nature 327:70-73).

After the targeting construct has been introduced into the animal cells, the cells in which the targeting construct has stably integrated into the genome can be selected. The choice of which one to use will generally depend upon the nature of the sequence that has been integrated. For example, if the targeting construct contains a selectable marker, then the integration of the targeting construct into the genome results in the stable acquisition of the selectable marker. In some situations the cells may be selected by virtue of a modification of the Immunoglobulin target gene. In other situations, a selectable phenotype may result from juxtaposition of a DNA sequence present on the targeting construct with DNA sequences present near the target DNA. For instance, integration of a promoterless antibiotic resistance gene at the target site may result in expression of the resistance gene based on transcriptional activity at the target site.

It is also possible, although not essential, to use the polymerase chain reaction (PCR) to screen cells in which homologous integration has occurred. In an advantageous application, one PCR primer is directed to DNA in the modification sequence and another primer is directed to DNA near the target locus that is outside but proximal to the target DNA, such that integration results in the creation of a genomic DNA sequence in which the primer binding sites are facing each other in relative juxtaposition. Amplification of this region produces DNA of a specific size confirming the presence of the targeted change to the genome. Homologous recombination can also be confirmed using standard DNA hybridization techniques, such as Southern blotting, to verify the presence of the integrated DNA in the desired genomic location.

An embodiment of the present invention includes using microcell mediate chromosome transfer (MMCT) as part of the knockout process. The introduction of limited numbers of chromosomes from one cell to another is well established in the literature. These methods rely on the generation of small cell-like structures, termed microcells, containing a limited amount of genetic material within a micronucleus that is itself surrounded by a rim of cytoplasm and an intact plasma membrane. As described previously, the chromosome to be transferred could comprise an avian chromosome having a knockout of an endogenous immunoglobulin locus by homologous recombination in a recombination-proficient cell such as a targeted knockout of the immunoglobulin heavy chain in a DT40 cell. Also, an engineered avian chromosome having human immunoglobulin loci added to the avian chromosome can be transferred by MMCT. In either case, the resulting embryonic stem cell contains three copies of a single avian chromosome, two of which are unmodified, endogenous chromosomes and one of which carries a modification that yields a functionally disrupted immunoglobulin gene incapable of mounting an immune response to an antigen challenge.

Microcells are produced from donor cells (preferably chicken fibroblasts that have selectable marker genes integrated into the chromosomes) by first exposing the cells to high concentrations of a mitotic inhibitor, such as colcemid for between 24 and 48 hours, at a concentration between 0.1 and 10 μl/ml. Exposure to the colcemid induces the cells to form micronuclei. The size of the micronuclei will determine the amount of genetic information available for transfer during microcell hybridization. After micronuclei have formed, the cells are enucleated by centrifugation in the presence of 5 to 20 μg/ml cytochalasin B in an appropriately buffered solution such as serum-free growth medium of phosphate-buffered saline (PBS). Enucleation of adherent cells is achieved by centrifuging the cells grown on a solid support, such that the supports are positioned vertically in centrifuge tubes containing the cytochalasin B solution. Non-adherent cells are enucleated by centrifugation through a Percoll gradient containing cytochalasin B, as described in more detail below. The micronuclei are recovered from the resulting pellet. The micronuclei are preferably size-selected to remove whole cells and to isolate micronuclei that contain approximately one chromosome. Size selection may be accomplished using sequential filtration through 8- and 5-μm filters. Alternatively, micronuclei can be size separated by unit gravity sedimentation on a linear 1-3% bovine serum albumin gradient, taking the upper fraction containing the smaller micronuclei.

Suitable cells for use in the present invention for targeted integration include immortalized avian cells which exhibit a high level of homologous recombination, such as DT40 line described above and LSCC-RP9. The choice of cell line would be evident to one skilled in the art given the selectable markers present in the chicken chromosome(s). This regard, the choice of the cell type of the recipient may be of importance when expression of the selectable marker is required. To fuse the microcells with the recipient cells, the preparation of microcells is incubated with the recipient cells for 10 to 15 minutes at 37° C. In the case of adherent cells, the microcells are preferably suspended in a solution of 100-200 μg/ml of phytohemagglutinin P and applied to monolayers of recipient cells to allow for agglutination. In the case of non-adherent recipient cells, the microcells and recipient cells are suspended together in a test tube. The microcells are fused to the recipient cells by a sixty second exposure of between 44 to 50% (wt/wt) polyethylene glycol (mW 1300-1600). The microcell hybrids are allowed to incubate overnight in nonselective medium. The cells are then place under selection in the appropriate medium to select for the presence of cells containing chromosome(s) from the donor cell having an integrated selectable marker gene.

The presence and identification of donor chromosomes in the microcell hybrids may be carried out using any number of well established methods. In the case of microcell hybrids containing chicken chromosomes, chicken chromosome may be detected by, for example, filter hybridization to detect chicken alleles or DNA markers.

### Example 1

### The Functional Disruption or Knockout of the Endogenous Avian Immunoglobulin Gene by Homologous Recombination in Avian Embryonic Stem Cells

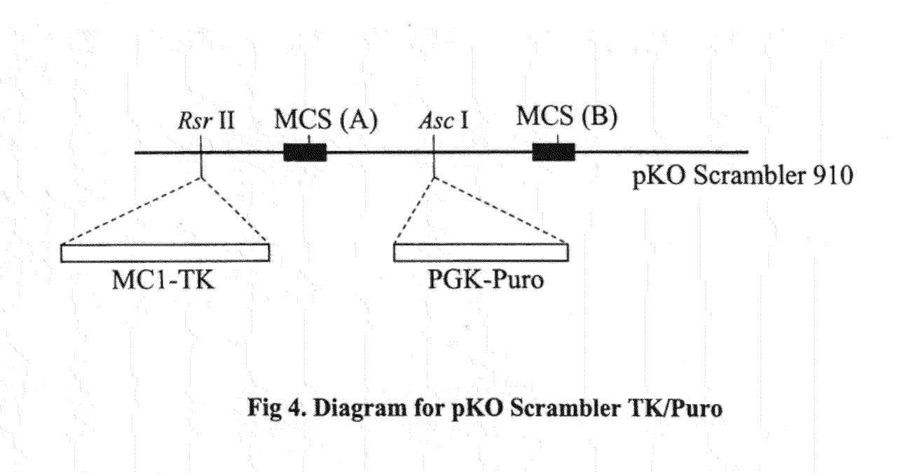

The puromycin expression cassette (1.5 Kb) was released from pKO SelectPuro (Stratagene) by Asc I digestion. Referring to FIG. 4, the resulting fragment was inserted into the Asc I site of pKO Scrambler 910 (Strategene), and verified by a Xho I digestion. Thymidine Kinase expression cassette (2.0 Kb) was released from pKO SelectTK (Stratagene) by Rsr II digestion. The resulting fragment was inserted into the Rsr II site of pKO Scrambler Puro, and verified by Sph I digest. The plasmid illustrated in FIG. 4 is the starting point for all the IgH and IgL targeting constructs.

### IgH KO

A genomic DNA fragment of chicken IgH (DJ-6) in germline configuration was obtained from Dr. Claude-Agnes Reynaud, University Paris. The 6.2 Kb EcoR I fragment contains coding sequences of the chicken IgH Dₓ, D₁, and J_{H}.

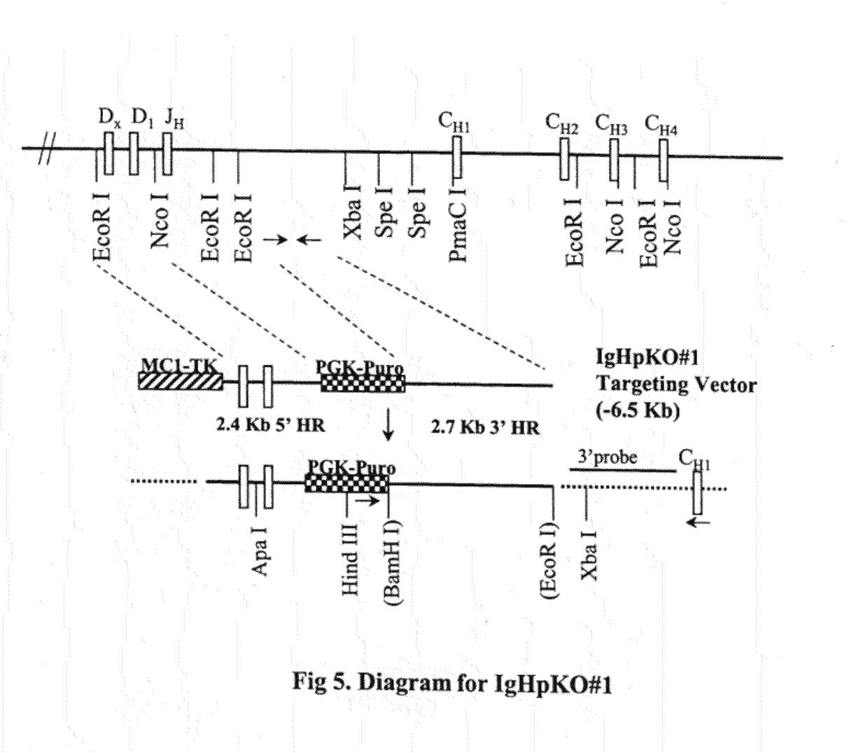

Referring to FIG. 5, the Dₓ, D₁, and J_{H} region of the chicken IgH (6.2 kb) was released from the vector portion of the DJ-6 plasmid by EcoR I. A subsequence Nco I partial digest of this fragment isolated a 2.2 Kb DNA fragment containing Dₓ and D₁. The fragment was blunted and inserted into Hpa I site (Scrambler A region) of pKO Scrambler TK/Puro to become the 5′ homologous region. This intermediate plasmid pKO TK/Puro-D_{X}-D₁ was verified with a Nco I digestion.

A 2687 bp fragment of chicken IgH switch and constant region (base 11-2697, Genebank #AB029075) was amplified from chicken genomic DNA. Primers Cu-1 (with BamH I site underlined) and Cu-2 (with EcoR I site underlined) were designed based on the above referenced sequence.

(SEQ ID NO.: 1)
Cu-1: 5′-CTCGGATCCCAACAAACGGCACTCGATAATT-3′
(SEQ ID NO.: 2)
Cu-2: 5′-CTCGAATTCTTCATTGACCTTCATTAACCGC-3′

The PCR product was cloned into pGEM-T easy vector (Promega) to form Cu2.7. The plasmid was confirmed by sequencing from the two ends.

The 2.7 Kb fragment of chicken IgH switch and constant region was released from Cu2.7 with BamH I and EcoR I. This fragment was inserted into the BamH I and EcoR I (Scrambler B region) of pKO TK/Puro-D_{X}-D₁ to form the 3′ homologous region. The resulting plasmid IgHpKO#1 (see FIG. 5) was verified by a BamH I and EcoR I double digestion. The clone was confirmed by sequencing from two ends for the presence of chicken IgH sequences. This plasmid can be linearized with Not I, Sal I or Sca I. After purification, the linearized DNA is ready for transfection.

### IgL KO

A genomic DNA fragment of chicken IgL (Cλ36SacI) in germline configuration was a gift from Dr. Claude-Agnes Reynaud, University Paris (EMBO J. 12:4615-23, 1993). The 10.5 Kb Sac I fragment contains coding sequences of chicken IgL V_{L}, J_{L}, and C_{L}, and 2.0 Kb and 3.8 Kb of 5′ and 3′ flanking sequences, respectively. Two IgL KO constructs are made.

### IgLpKO#3

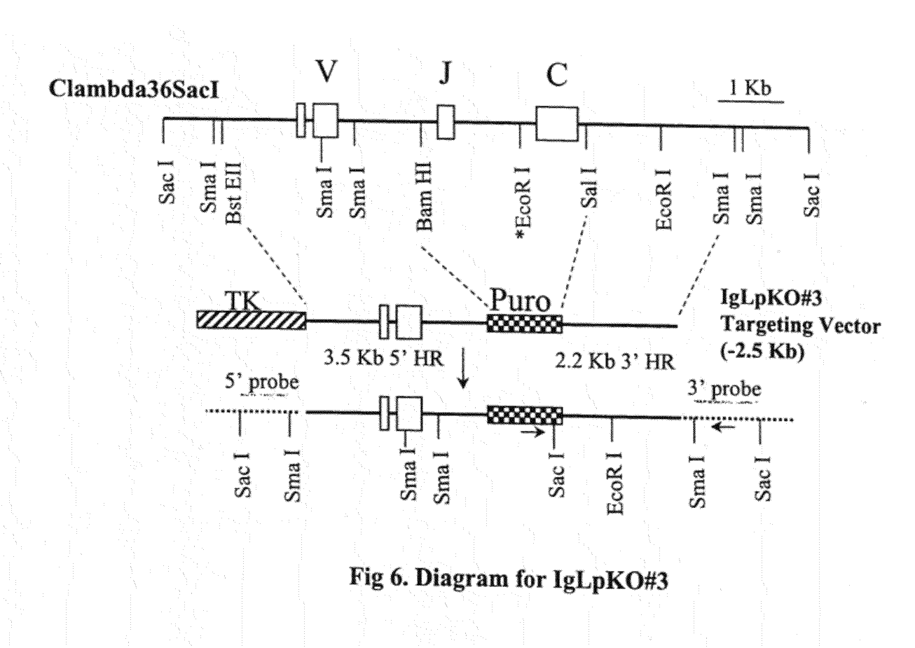

Referring to FIG. 6, a 2.0 Kb fragment of chicken IgL 3′ flanking sequences was released from Cλ36SacI by Sal I and Sma I double digestion. The fragment was then blunted and inserted into the Sma I site (Scrambler B region) of pKO scrambler TK/Puro to form the 3′ homologous region. This intermediate plasmid 3′IgLpKO-TK/puro was verified with EcoR I digestion for correct orientation.

The chicken IgL fragment (10.5 Kb) was released from Cλ36SacI by Sac I digestion. A subsequent BstE II and BamH I double digestion isolated the fragment of IgL V region (3.5 Kb). The fragment was then blunted and inserted into the Hpa I site (Scrambler A region) of the 3′IgLpKO-TK/Puro to become the 5′ homologous region. This plasmid IgLpKO#3 (see FIG. 6) was verified with Sma I and Rsr II double digestion for correct orientation. The plasmid was further confirmed by sequencing from the two ends. The plasmid can be linearized with Sal I and purified for transfection.

### IgLpKO#13

In another embodiment, a 2396 bp fragment of chicken IgL V region (base 24-2419, Genebank #M24403) was amplified from chicken genomic DNA. Primers CiGL5A (with Hpa I sequence underlined) and CiGl5B (with Hpa I sequence underlined) were designed based on the above-referenced sequence.

(SEQ ID NO.: 3)
cIgL5A: 5′-CTCGTTAACGATGTTGTACTGAGGGATGTGG-3′
(SEQ ID NO.: 4)
cIgL5B: 5′-CTCGTTAACCGGTGAACAAGGATGTTCAGTA-3′

The PCR product was cloned into pGEM-T easy vector (Promega). The resulting plasmid 5′IgL was confirmed by sequencing from the two ends. The 2.4 Kb V_{L} region of chicken IgL was released from 5′ IgL plasmid by Hpa I digestion. The fragment was then inserted into Hpa I site (Scrambler A region) of pKO scrambler TK/Puro. This intermediate plasmid 5′IgLpKO-TK/Puro was verified with a KpnI or NsiI digestion for correct orientation.

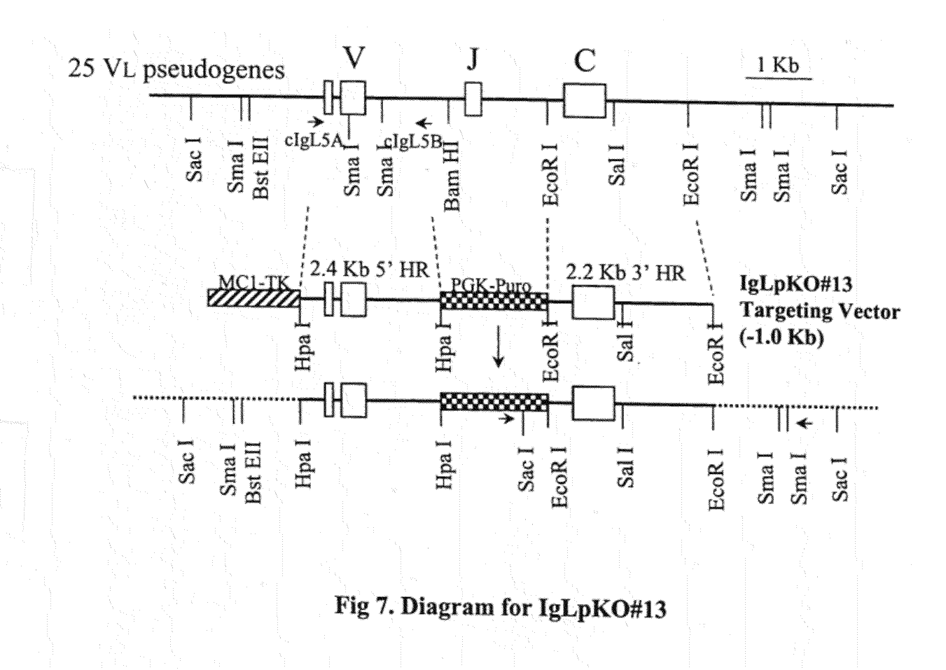

Referring to FIG. 7, a 2.8 Kb fragment of chicken IgL C region was released from Cλ36SacI by EcoR I digestion. The fragment was then inserted into the EcoR I site (Scrambler B region) of 5′IgLpKO-TK/Puro to form the 3′ homologous region. This plasmid IgLpKO#13 (see FIG. 7) was verified with Nsi I digestion for correct orientation and further confirmed by sequencing from the two ends. IgLpKO#13 can be linearized with Not I and purified for transfection.

One representative hybrid from each cross was chosen for further analysis. FISH and hybridization clones were used to determine if the transfer was successful. To determine whether the targeting locus was intact in the microcell/DT40 hybrids, Southern blot analysis was performed using six different probes spanning approximately 200 kb.

The transgenomic microcell/DT40 hybrids were micronucleated in 0.015 μg/ml colcemid for 48 hours and enucleated by centrifugation through Percoil as described above. Microcells were fused to 2−3×10⁷ recipient ES cells as above. The fusions were incubated as a pool in non-selective media for 24 hours, then plated at clonal density into 8×96-well microtiter plates in 0.2 ml/well of medium containing 500 μg/ml hygormycin B plus 40 μg/ml DAP. After 3-4 weeks, hybrid clones were picked individually and maintained in the same medium containing 0.25 μg/ml hygromycin B. The genotypes were determined by FISH and hybridization clones were used to determine if the Immunoglobulinclone was transferred. To determine whether the targeting locus was intact in the microcell/DT40 hybrids, Southern blot analysis was performed using six different probes spanning approximately 200 kb. As above, the cells were then used to produce chimeric chickens.

### Example 2

### Chromosome Transfer Using DT40 Cells

DT40 cells containing chromosome of interest, such as an avian chromosome 15 lacking the immunoglobulin heavy chain locus, are grown up in DMEM/10% FBS/5% chicken serum/10% tryptose phosphate broth/0.1 μm β-mercaptoethanol/2 mM glutamine/pen-strep and appropriate selection drug. 1.6×10⁸ cells are obtained and demecolcine is added to 0.01 μg/ml final concentration (1:1000) and maintained for 48-72 hours. Fresh Percoll (Pharmacia) is prepared by equilibrating with NaCl to a final concentration of 150 mM and Hepes buffer, pH 7.0, to a final concentration of 50 mM. 17.5 ml of equilibrated Percoll is added to 6 50-ml Oak Ridge polycarbonate tubes (Nalgene). DT40 cells are harvested by pelleting (save 500 μl for Hoechst staining) The cell population is resuspended in 105 ml DMEM/10% FBS/20 μg/ml cytochalasin B (1.3×106 cells/ml) and cell clumps are broken up by trituration before loading onto the gradient. 210 μl of Demecolcine are added to cells before combining with Percoll for a final concentration of 0.01 μg/ml and 17.5 mls of cells are added to each Percoll tube and mixed well by inverting. The resulting composition is centrifuged in an Avanti centrifuge with a JA-25.50 rotor at 19,415 rpm (30,924 g) for 80 min at 32° C. (no brake). Material is pooled from about 2 cm below the top of each tube to the region just above the Percoll pellet and centrifuged again at 2000 g for 5 minutes. The microcells are resuspended in 50 ml DMEM (no serum) by vigorous pipetting. This step is repeated a total of 3 times to rid the suspension of all Percoll. The cells are filtered sequentially through 8 μm, then 5 μm, then 3 μm filters yielding 3-9×10⁷ microcells. 500 μl are saved for Hoechst staining 10⁷ recipient cells are harvested, washed three times with DMEM (no serum) and resuspended in 5 ml DMEM, prior to being combined with microcells and centrifuged at 1250 rpm for 5 minutes to remove supernatant. The cells and microcells are resuspended in 5 ml DMEM+100 μg/ml phytohemagglutinin P for 10 minutes at room temp, and then spun down. The pellet is dispersed by tapping and then slowly dripping 0.3 ml of a PEG solution (0.25 g sterile PEG in glass vial, melt, add 50 μl DMSO, 0.3 ml DMEM). 1 ml DMEM is immediately added in a dropwise fashion while swirling, then another 1 ml, then 7 ml with gentle swirling. The resulting mixture is centrifuged at 1000 rpm for 5 minutes, rinsed in DMEM, and re-centrifuged. The pellet is resuspended in regular growth media and plated. After 24 hours the media is replaced with selective media.

### Example 3

### The Functional Replacement of Endogenous Avian Immunoglobulin Heavy Chain Genes with Unrearranged Human Loci

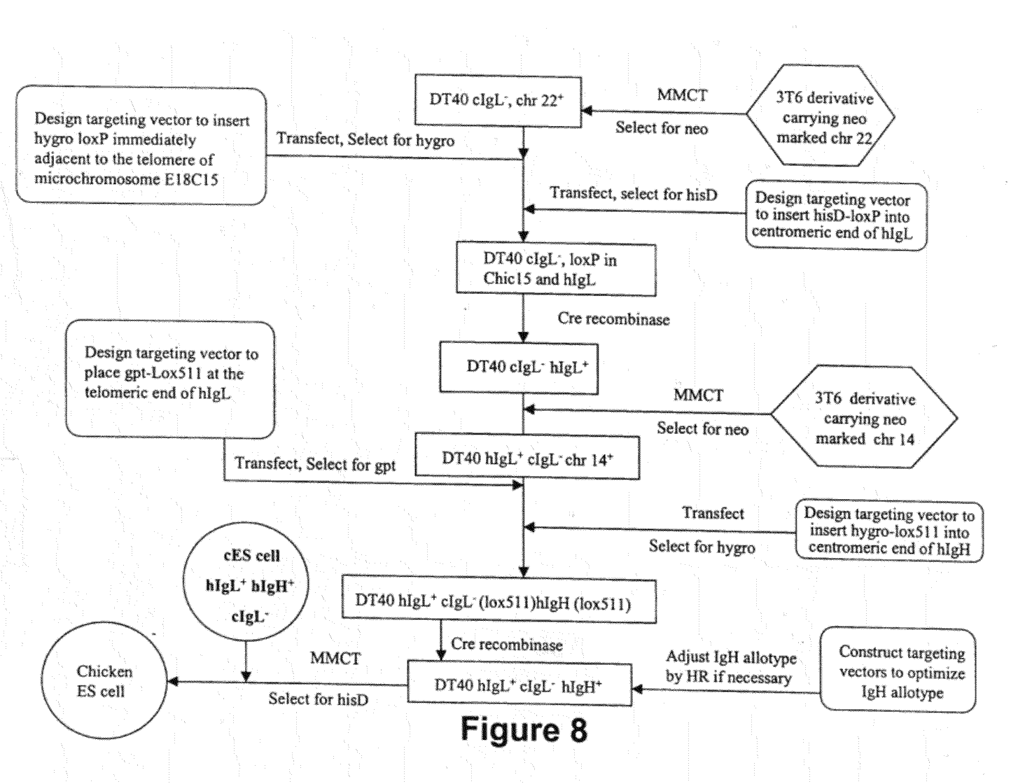

In another embodiment, the engineered chromosome contains a locus that is desired to be deleted and that is proximate to the telomere of an identified chromosome. Referring to FIG. 8, to achieve the deletion, a site specific recombination site is inserted centromeric of the locus such that the entire locus is deleted to yield the engineered chromosome. Alternatively, the recombination site can be placed within the locus such that recombination renders the locus non-functional. In this embodiment, the construct used to create the engineered chromosome may contain exogenous DNA thereby creating a chimeric chromosome that is comprised almost entirely of a native chromosome but with a exogenous segment of DNA at the telomeric region of the chromosome. In a particularly preferred embodiment, the endogenous chicken immunoglobulin heavy chain gene is located at a site that is proximate to the telomere of chicken chromosome 15. The location of the heavy chain locus at the telomeric end of the chromosome provides the ability to render the locus non-functional or to delete the entire locus through site-specific recombination.

The ability to target a locus for deletion by this method is a function of the necessity to the organism of the region of DNA that is telomeric to locus. Depending on the organism, if the telomeric DNA is not necessary for the survival of the organism, such that the deletion of all DNA telomeric of the locus results in a non-lethal mutation, then the functional disruption of the gene may be achieved by a recombination event that is centromeric of the locus. Referring again to FIG. 8, in this embodiment, the construction of the engineered chromosome of the invention includes the insertion of a recombination site such as a Lox site centromeric of a region of DNA comprised of the endogenous immunoglobulin heavy chain gene or directly within a recombination-competent site such as the J region. In a preferred version of this embodiment, at least one site-specific recombination site is inserted at a point such that deletion of all of the DNA telomeric of the site renders the chicken immunoglobulin heavy chain locus non-functional or non-existent. Subsequently, a construct containing a complimentary recombination site attached to a segment of exogenous DNA comprised of at least one human immunoglobulin locus is inserted into the cell. Preferably, the “construct” in this context is the entire human chromosome 2, 14, or 22 containing a recombination site centromeric of the human heavy or light chain immunoglobulin gene respectively. When the construct is introduced into the DT40 cell under conditions that facilitate site-specific recombination, the human immunoglobulin locus replaces the avian immunoglobulin heavy chain locus via recombination of the Lox site. Thus, when the modified avian chromosome containing the Lox recombination site and the human chromosome with an immunoglobulin locus and a complementary Lox site are combined in the DT40 cell under conditions causing recombination, the construct replaces all of the endogenous DNA that is telomeric of the recombination site and the endogenous chicken immunoglobulin heavy chain gene is deleted and replaced with a construct containing human immunoglobulin loci.

The first construct, when integrated into the avian chromosome may also contain a second recombination site that is telomeric of the unrearranged human immunoglobulin locus. In this embodiment, at least two human immunoglobulin loci may be inserted into the modified avian chromosome 15. Because the human immunoglobulin heavy chain locus is known to be telomeric at chromosome 14, it is preferred that the deletion or functional disruption of the avian immunoglobulin heavy chain locus be achieved with a construct comprised of the human immunoglobulin light chain locus as a first step. Thus, the first construct is human chromosome 14 with an appropriate recombination site. When a first recombination step occurs, the modified chromosome is comprised of the native avian chromosome 15 absent the immunoglobulin heavy chain locus, but having the human immunoglobulin light chain locus at the telomeric end. As a second stage, a dissimilar recombination site, such as a second Lox site is inserted at the telomeric end of the human immunoglobulin light chain locus that is now part of the modified avian chromosome.

Alternatively, two dissimilar recombinations sites, such as a Lox P and Lox 511 site can be simultaneously inserted into human chromosome 14 such that the first recombination step provides two dissimilar recombination sites. By either approach, the chimeric chromosome has a second recombination site at a telomeric end. Thus, in this intermediate configuration, the modified, engineered avian chromosome 15 contains a first recombination site centromeric of the human immunoglobulin light chain locus and a second dissimilar recombination site telomeric of the locus. This configuration is suited for reaction with a second construct containing the portion of DNA from human chromosome 14 comprising the human immunoglobulin heavy chain locus. Placed under conditions suitable for recombination of the second (but not the first) recombination site, the human immunoglobulin heavy chain locus is integrated into avian chromosome 15 at a site telomeric of the human immunoglobulin light chain lamda locus. In a similar fashion, a second human immunoglobulin light chain locus may be integrated into avian chromosome 15 in an orientation compatible with the existing loci locus.

The modified avian chromosome of the invention has several advantages and unique features compared to existing avian chromosomes and other genetic modifications that have been made for the production of human immunoglobulins. In the configuration described above, the modified chicken chromosome 15 is capable of expressing human immunoglobulins from an avian chromosome. Moreover, in the preferred embodiment, the modified avian chromosome expresses both heavy and light chains of the human immunoglobulin repertoire. Thus, contrary to the endogenous human immunoglobulin gene loci, both chains are expressed loci on the same chromosome. Furthermore, the modified avian chromosome contains human immunoglobulin DNA that is both integral to the avian chromosome and is oriented in germline configuration and, therefore exists in an unrearranged state that, when successfully used in a transgenic application, results in human immunoglobulin DNA integral to the avian chromosome that is capable of responding to antigen challenge by rearranging to encode immunoglobulin molecules specific for the antigen. Moreover, the deletion of the entire endogenous chicken heavy chain locus avoids the potential for trans-chromosomal switching sometimes observed in murine transgenic immunoglobulin production models. Because the heavy chain immunoglobulin gene disruption in murine models is a deletion of a recombination-competent locus such as a J segment, that prevents immunoglobulin production by preventing V-D-J joining prior to combination of the V-D-J light chain subassembly with an immunoglobulin heavy chain, the endogenous murine constant region DNA remains in place even in the knockout animal. In the mouse model, the remaining murine constant region is available to join with a rearranged V-D-J subunit of the exogenous human DNA, thus resulting in a chimeric antibody that is partially human and partially murine. The strategy described above eliminates this possibility by deleting the endogenous heavy chain immunoglobulin locus.

There will be various modifications, improvements, and applications of the disclosed invention that will be apparent to those of skill in the art, and the present application encompasses such embodiments to the extent allowed by law. Although the present invention has been described in the context of certain preferred embodiments, the full scope of the invention is not so limited, but is in accord with the scope of the following claims. All references, patents, or other publications are specifically incorporated by reference herein.

What is claimed:

1. An avian embryonic stem cell comprising a functional disruption of an immunoglobulin gene.

2. The cell of claim 1 wherein the genome of the cell is comprised of a targeting vector integrated into the chicken immunoglobulin gene.

3. The cell of claim 2 wherein the targeting vector has at least two separate regions that are each homologus to the chicken immunoglobulin gene.

4. The cell of claim 3 wherein the positive selection marker is located between the regions of homology.

5. The cell of claim 2 wherein the immunoglobulin gene is a heavy chain.

6. The cell of claim 2 wherein the immunoglobulin gene is a light chain.


SEQUENCE LISTING (4 sequences)

SEQ ID NO: 1 - DNA, 31 bp, Artificial Sequence
  Primer Cu-1
ctcggatcccaacaaacggcactcgataatt

SEQ ID NO: 2 - DNA, 31 bp, Artificial Sequence
  Primer Cu-2
ctcgaattcttcattgaccttcattaaccgc

SEQ ID NO: 3 - DNA, 31 bp, Artificial Sequence
  Primer cIgL5A
ctcgttaacgatgttgtactgagggatgtgg

SEQ ID NO: 4 - DNA, 31 bp, Artificial Sequence
  Primer cIgL5B
ctcgttaaccggtgaacaaggatgttcagta